(19) United States
(12) Patent Application Publication
(10) Pub. No.: US 2018/0269709 A1
(43) Pub. Date: Sep. 20, 2018

(54) ADJUSTABLE-LENGTH WIRELESS POWER TRANSMITTER

(71) Applicant: QUALCOMM Incorporated, San Diego, CA (US)

(72) Inventors: Mark White, II, San Diego, CA (US); William Henry Von Novak, III, San Diego, CA (US); Seong Heon Jeong, San Diego, CA (US); Cody Wheeland, San Diego, CA (US)

(21) Appl. No.: 15/459,881

(22) Filed: Mar. 15, 2017

(51) Int. Cl. H02J 7/02; H02J 50/10; H02J 50/40; H02J 50/90; A61N 1/05

(52) U.S. Cl. CPC H02J 7/025; H02J 50/10; A61N 1/05; H02J 50/90; H02J 50/40

(57) ABSTRACT

A wireless charging device includes: a base configured to be worn by a user; and a coil attached to the base and comprising an electrically conductive material shaped to produce a magnetic field to convey power wirelessly to a receiver in response to receiving power, the coil including multiple turns each having a turn length with at least one of the multiple turns having an adjustable turn length, the multiple turns being disposed along a common axis such that each of the multiple turns is disposed around the axis for the respective turn length of the turn.

### TECHNICAL FIELD

The disclosure relates generally to wireless power delivery to electronic devices, and in particular to adjustable-length power transmitters for wireless power transfer.

### BACKGROUND

An increasing number and variety of electronic devices are powered via rechargeable batteries. Such devices include mobile phones, portable music players, laptop computers, tablet computers, computer peripheral devices, communication devices (e.g., BLUETOOTH devices), digital cameras, hearing aids, and the like. While battery technology has improved, battery-powered electronic devices increasingly require and consume greater amounts of power. As such, these devices frequently require recharging. Rechargeable devices are often charged via wired connections that require cables or other similar connectors that are physically connected to a power supply. Cables and similar connectors may sometimes be inconvenient or cumbersome and have other drawbacks. Wireless power charging systems may allow users to charge and/or power electronic devices without physical, electro-mechanical connections, thus simplifying the use of the electronic device.

### SUMMARY

The following detailed description and accompanying drawings provide a better understanding of the nature and advantages of the disclosure.

An example of a wireless charging device includes: a base configured to be worn by a user; and a coil attached to the base and comprising an electrically conductive material shaped to produce a magnetic field to transmit power wirelessly to a receiver in response to receiving power, the coil including multiple turns each having a turn length with at least one of the multiple turns having an adjustable turn length, the multiple turns being disposed along an axis such that each of the multiple turns is disposed around the axis for the respective turn length of the turn.

Another example of a wireless charging device includes: transmitting means for wirelessly transmitting power, the transmitting means including an input port and a return port, the input port and the return port being configured to electrically couple to a power source; and housing means for housing the transmitting means and for positioning the transmitting means around of a first portion of a first user's body of a first perimeter length, where the transmitting means are further for extending a turn length of a conductor coupling the input port to the return port for the transmitting means to be positioned around a second portion of a second user's body of a second perimeter length that is greater than the first perimeter length.

An example of a method of providing wireless power to an implant includes: wrapping a transmitter coil substantially around a portion of a user; adjusting a turn length of the transmitter coil; and energizing the transmitter coil to produce a magnetic field along a length of the portion of the user.

### BRIEF DESCRIPTION OF THE DRAWINGS

Drawing elements that are common among the following figures may be identified using the same reference numerals.

With respect to the discussion to follow and in particular to the drawings, the particulars shown represent examples for purposes of illustrative discussion, and are presented in the cause of providing a description of principles and conceptual aspects of the disclosure. In this regard, no attempt is made to show implementation details beyond what is needed for a fundamental understanding of the disclosure. The discussion to follow, in conjunction with the drawings, makes apparent to those of skill in the art how embodiments in accordance with the disclosure may be practiced.

FIG. 1 is a functional block diagram of an example of a wireless power transfer system.

FIG. 2 is a functional block diagram of an example of another wireless power transfer system.

FIG. 3 is a schematic diagram of an example of a portion of transmit circuitry or receive circuitry of the system shown in FIG. 2.

FIG. 4 is a simplified diagram of a person wearing adjustable-length wireless power transmitters.

FIG. 5 is perspective view of an example of an adjustable-length wireless power transmitter shown in FIG. 4.

FIG. 6 is a side, partially-cut-away, view of a portion of the transmitter shown in FIG. 5.

FIG. 7 is a side, partially-cut-away, view of an input and a return port of the transmitter shown in FIG. 5.

FIGS. 8-9 are example configurations of coils and connector configurations at intermediate ends of the transmitter shown in FIG. 5.

FIG. 10 is a simplified side cut-away view of connectors and extendable electrical segments of an example extension of the transmitter shown in FIG. 5.

FIG. 11 is a simplified side view of an example connector of the transmitter shown in FIG. 5.

FIG. 12 is a simplified side view of another example connector of the transmitter shown in FIG. 5.

FIG. 13 is a perspective view of another example of an adjustable-length wireless power transmitter shown in FIG. 4.

FIG. 14 is a side cut-away view of intermediate ends and a cleat of the transmitter shown in FIG. 13.

FIG. 15 is a side, partially-cut-away, view of an alternative portion of the transmitter shown in FIG. 5.

FIG. 16 is a block flow diagram of a method of providing wireless power to an implant.

### DETAILED DESCRIPTION

Wireless power transfer may refer to transferring any form of energy associated with electric fields, magnetic fields, electromagnetic fields, or otherwise from a transmitter to a receiver without physical electrical conductors attached to and connecting the transmitter to the receiver to deliver the power (e.g., power may be transferred through free space). The power output into a wireless field (e.g., a magnetic field or an electromagnetic field) may be received, captured by, or coupled to by a power receiving element to achieve power transfer. The transmitter transfers power to the receiver through a wireless coupling of the transmitter and receiver.

Techniques are discussed herein for adaptively transmitting wireless power. For example, an adjustable-length transmitter is provided that has an adjustable length coil for magnetically coupling power wirelessly to a receiver. The coil is preferably a multi-turn coil. The transmitter may be disposed on a carrier such as an animal body, e.g., a human body, and may be particularly adapted for wirelessly transferring power to an implant inside the carrier. For example, a transmitter system containing a multi-turn transmitter coil may be wrapped around a torso, a waist, or a limb of a person and a length of the transmitter coil adjusted based on a perimeter of the torso, waist, or limb of the person. Various mechanisms may be employed for adjusting a length of the coil. For example, the transmitter coil may be separable, and one or more extensions may be used to connect ends of the transmitter coil to change the length of the coil, e.g., the length of one or more, and preferably all, of the turns of the coil. As another example, a corset-type transmitter may be placed around a carrier such as a person and cinched to adapt a length of the transmitter to a perimeter length of the person. Other examples are within the scope of the disclosure, some of which are discussed below.

Items and/or techniques described herein may provide one or more of the following capabilities, as well as other capabilities not mentioned. Wireless power may be provided efficiently to implants in carriers of different sizes, at various orientations, and/or at various depths within a carrier. A wireless power transmitter may be wearable by a user and adjustable to improve energy transfer and inhibit slippage or other movement of the transmitter. A magnetic field for wireless power transfer may be produced along an axis of a region of a user.

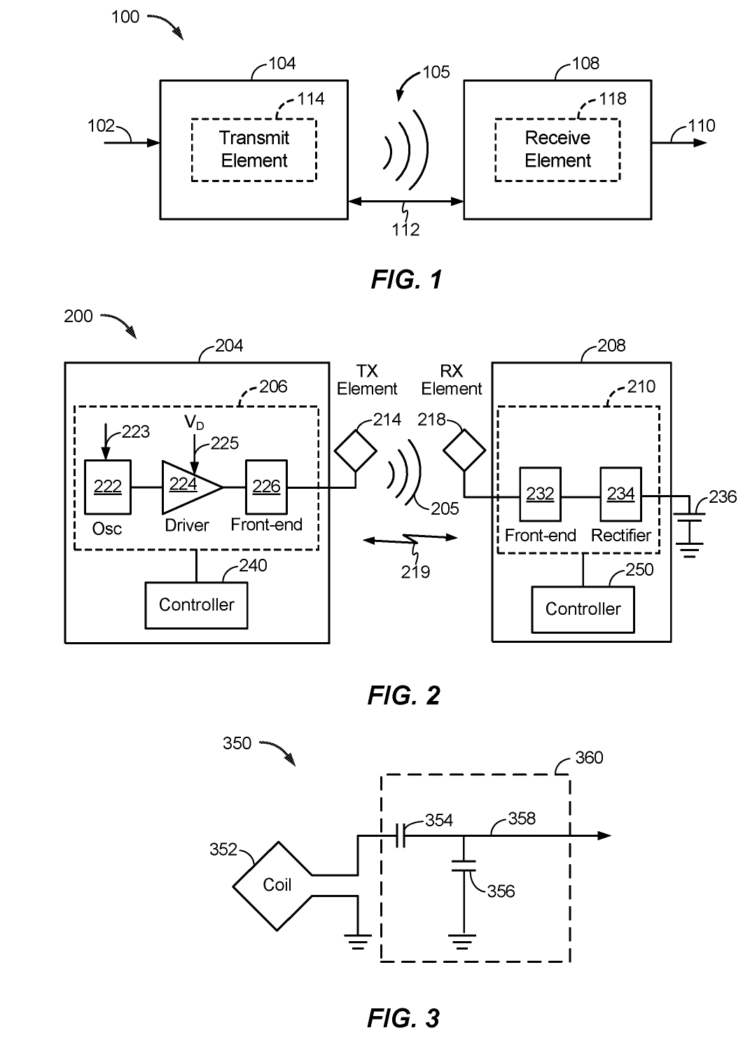

FIG. 1 is a functional block diagram of an example of a wireless power transfer system 100. Input power 102 may be provided to a transmitter 104 from a power source (not shown in this figure) to generate a wireless (e.g., magnetic or electromagnetic) field 105 for performing energy transfer. A receiver 108 may couple to the wireless field 105 and generate output power 110 for storing or consumption by a device (not shown in this figure) that is coupled to receive the output power 110. The transmitter 104 and the receiver 108 are separated by a non-zero distance 112. The transmitter 104 includes a power transmitting element 114 configured to transmit/couple energy to the receiver 108. The receiver 108 includes a power receiving element 118 configured to receive or capture/couple energy transmitted from the transmitter 104.

The transmitter 104 and the receiver 108 may be configured according to a mutual resonant relationship. When the resonant frequency of the receiver 108 and the resonant frequency of the transmitter 104 are substantially the same, transmission losses between the transmitter 104 and the receiver 108 are reduced compared to the resonant frequencies not being substantially the same. As such, wireless power transfer may be provided over larger distances when the resonant frequencies are substantially the same. Resonant inductive coupling techniques allow for improved efficiency and power transfer over various distances and with a variety of inductive power transmitting and receiving element configurations.

The wireless field 105 may correspond to the near field of the transmitter 104. The near field corresponds to a region in which there are strong reactive fields resulting from currents and charges in the power transmitting element 114 that do not significantly radiate power away from the power transmitting element 114. The near field may correspond to a region that up to about one wavelength, of the power transmitting element 114. Efficient energy transfer may occur by coupling a large portion of the energy in the wireless field 105 to the power receiving element 118 rather than propagating most of the energy in an electromagnetic wave to the far field.

The transmitter 104 may output a time-varying magnetic (or electromagnetic) field with a frequency corresponding to the resonant frequency of the power transmitting element 114. When the receiver 108 is within the wireless field 105, the time-varying magnetic (or electromagnetic) field may induce a current in the power receiving element 118. As described above, with the power receiving element 118 configured as a resonant circuit to resonate at the frequency of the power transmitting element 114, energy may be efficiently transferred. An alternating current (AC) signal induced in the power receiving element 118 may be rectified to produce a direct current (DC) signal that may be provided to charge an energy storage device (e.g., a battery) or to power a load.

FIG. 2 is a functional block diagram of an example of a wireless power transfer system 200. The system 200 includes a transmitter 204 and a receiver 208. The transmitter 204 (also referred to herein as power transmitting unit, PTU) is configured to provide power to a power transmitting element 214 that is configured to transmit power wirelessly to a power receiving element 218 that is configured to receive power from the power transmitting element 214 and to provide power to the receiver 208. Despite their names, the power transmitting element 214 and the power receiving element 218, being passive elements, may transmit and receive power and communications.

The transmitter 204 includes the power transmitting element 214, transmit circuitry 206 that includes an oscillator 222, a driver circuit 224, and a front-end circuit 226. The power transmitting element 214 is shown outside the transmitter 204 to facilitate illustration of wireless power transfer using the power receiving element 218. The oscillator 222 may be configured to generate an oscillator signal at a desired frequency that may adjust in response to a frequency control signal 223. The oscillator 222 may provide the oscillator signal to the driver circuit 224. The driver circuit 224 may be configured to drive the power transmitting element 214 at, for example, a resonant frequency of the power transmitting element 214 based on an input voltage signal (VD) 225. The driver circuit 224 may be a switching amplifier configured to receive a square wave from the oscillator 222 and output a sine wave.

The front-end circuit 226 may include a filter circuit configured to filter out harmonics or other unwanted frequencies. The front-end circuit 226 may include a matching circuit configured to match the impedance of the transmitter 204 to the impedance of the power transmitting element 214. As will be explained in more detail below, the front-end circuit 226 may include a tuning circuit to create a resonant circuit with the power transmitting element 214. As a result of driving the power transmitting element 214, the power transmitting element 214 may generate a wireless field 205 to wirelessly output power at a level sufficient for charging a battery 236, or powering a load.

The transmitter 204 further includes a controller 240 operably coupled to the transmit circuitry 206 and configured to control one or more aspects of the transmit circuitry 206, or accomplish other operations relevant to managing the transfer of power. The controller 240 may be a micro-controller or a processor. The controller 240 may be implemented as an application-specific integrated circuit (ASIC). The controller 240 may be operably connected, directly or indirectly, to each component of the transmit circuitry 206. The controller 240 may be further configured to receive information from each of the components of the transmit circuitry 206 and perform calculations based on the received information. The controller 240 may be configured to generate control signals (e.g., signal 223) for each of the components that may adjust the operation of that component. As such, the controller 240 may be configured to adjust or manage the power transfer based on a result of the operations performed by the controller 240. The transmitter 204 may further include a memory (not shown) configured to store data, for example, such as instructions for causing the controller 240 to perform particular functions, such as those related to management of wireless power transfer.

The receiver 208 (also referred to herein as power receiving unit, PRU) includes the power receiving element 218, and receive circuitry 210 that includes a front-end circuit 232 and a rectifier circuit 234. The power receiving element 218 is shown outside the receiver 208 to facilitate illustration of wireless power transfer using the power receiving element 218. The front-end circuit 232 may include matching circuitry configured to match the impedance of the receive circuitry 210 to the impedance of the power receiving element 218. As will be explained below, the front-end circuit 232 may further include a tuning circuit to create a resonant circuit with the power receiving element 218. The rectifier circuit 234 may generate a DC power output from an AC power input to charge the battery 236, as shown in FIG. 3. The receiver 208 and the transmitter 204 may additionally communicate on a separate communication channel 219 (e.g., BLUETOOTH, ZIGBEE, cellular, etc.). The receiver 208 and the transmitter 204 may alternatively communicate via in-band signaling using characteristics of the wireless field 205.

The receiver 208 may be configured to determine whether an amount of power transmitted by the transmitter 204 and received by the receiver 208 is appropriate for charging the battery 236. The transmitter 204 may be configured to generate a predominantly non-radiative field with a direct field coupling coefficient (k) for providing energy transfer. The receiver 208 may directly couple to the wireless field 205 and may generate an output power for storing or consumption by a battery (or load) 236 coupled to the output or receive circuitry 210.

The receiver 208 further includes a controller 250 that may be configured similarly to the transmit controller 240 as described above for managing one or more aspects of the wireless power receiver 208. The receiver 208 may further include a memory (not shown) configured to store data, for example, such as instructions for causing the controller 250 to perform particular functions, such as those related to management of wireless power transfer.

As discussed above, transmitter 204 and receiver 208 may be separated by a distance and may be configured according to a mutual resonant relationship to try to minimize transmission losses between the transmitter 204 and the receiver 208.

FIG. 3 is a schematic diagram of an example of a portion of the transmit circuitry 206 or the receive circuitry 210 of FIG. 2. While a coil, and thus an inductive system, is shown in FIG. 3, other types of systems, such as capacitive systems for coupling power, may be used, with the coil replaced with an appropriate power transfer (e.g., transmit and/or receive) element. As illustrated in FIG. 3, transmit or receive circuitry 350 includes a power transmitting or receiving element 352 and a tuning circuit 360. The power transmitting or receiving element 352 may also be referred to or be configured as an antenna such as a “loop” antenna. The term “antenna” generally refers to a component that may wirelessly output energy for reception by another antenna and that may receive wireless energy from another antenna. The power transmitting or receiving element 352 may also be referred to herein or be configured as a “magnetic” antenna, such as an induction coil (as shown), a resonator, or a portion of a resonator. The power transmitting or receiving element 352 may also be referred to as a coil or resonator of a type that is configured to wirelessly output or receive power. As used herein, the power transmitting or receiving element 352 is an example of a “power transfer component” of a type that is configured to wirelessly output and/or receive power. The power transmitting or receiving element 352 may include an air core or a physical core such as a ferrite core (not shown).

When the power transmitting or receiving element 352 is configured as a resonant circuit or resonator with tuning circuit 360, the resonant frequency of the power transmitting or receiving element 352 may be based on the inductance and capacitance. Inductance may be simply the inductance created by a coil and/or other inductor forming the power transmitting or receiving element 352. Capacitance (e.g., a capacitor) may be provided by the tuning circuit 360 to create a resonant structure at a desired resonant frequency. As a non-limiting example, the tuning circuit 360 may comprise a capacitor 354 and a capacitor 356, which may be added to the transmit or receive circuitry 350 to create a resonant circuit.

The tuning circuit 360 may include other components to form a resonant circuit with the power transmitting or receiving element 352. As another non-limiting example, the tuning circuit 360 may include a capacitor (not shown) placed in parallel between the two terminals of the circuitry 350. Still other designs are possible. For example, the tuning circuit in the front-end circuit 226 may have the same design (e.g., 360) as the tuning circuit in the front-end circuit 232. Alternatively, the front-end circuit 226 may use a tuning circuit design different than in the front-end circuit 232.

For power transmitting elements, the signal 358, with a frequency that substantially corresponds to the resonant frequency of the power transmitting or receiving element 352, may be an input to the power transmitting or receiving element 352. For power receiving elements, the signal 358, with a frequency that substantially corresponds to the resonant frequency of the power transmitting or receiving element 352, may be an output from the power transmitting or receiving element 352. Although aspects disclosed herein may be generally directed to resonant wireless power transfer, persons of ordinary skill will appreciate that aspects disclosed herein may be used in non-resonant implementations for wireless power transfer.

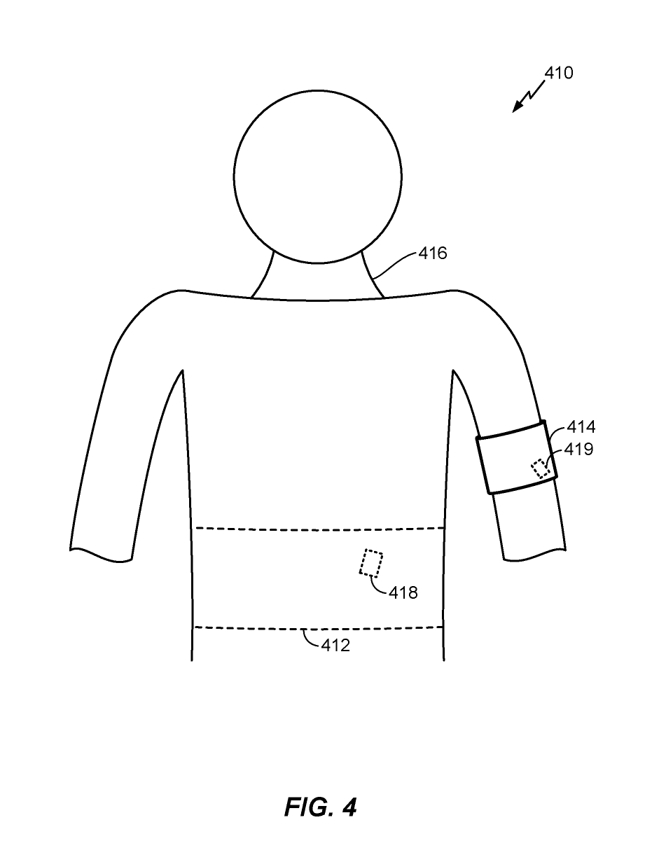

Referring to FIG. 4, a wireless power environment 410 includes two examples of wearable transmitter systems, here, a belt transmitter system 412 and an arm cuff transmitter system 414. Each of the transmitters systems 412, 414 may be referred to simply as a system or a transmitter. The transmitter 412 is configured to be positioned and sized (including being repositioned and re-sized) to be able to provide power wirelessly to an implant 418, and the transmitter 414 is configured to be positioned and sized (including being repositioned and re-sized) to be able to provide power wirelessly to an implant 419. The transmitters 412, 414 are configured to provide sufficient power to the implants 418, 419 to operate and/or charge the implants 418, 419. The transmitters 412, 414 may be positioned and sized, using techniques discussed herein, depending upon a location of an implant to be charged/operated. The belt transmitter system 412 is shown as being disposed or positioned about a midsection (e.g., a lower torso portion) of a user 416, although other configurations and locations are possible, such as being disposed lower or higher on the user 416, being thinner than as shown, etc. The systems 412, 414 may each be configured to include a multi-turn coil as the transmitter element 214 (FIG. 2), with multiple coils being disposed along an axis that is shared with the user 416. For example, when disposed for use, the system 412 may have coils disposed along a common axis with a torso of the user 416, and the system 414 may have coils disposed along a common axis of an arm of the user 416. Having the coils disposed along a common axis (e.g., layered on top of each other and around the axis) with a portion of the user 416 does not require that the coils are centered on the axis, but the coils are disposed around the axis. Other sizes, shapes, and applications of transmitter systems may be used, for example, a system disposed about a forearm of the user 416, a system disposed around a thigh of the user 416, a system disposed around a calf of the user 416, etc. In these examples, the transmitter system preferably includes a housing for conductive coils with the housing being made of a flexible material that may be wrapped around and possibly conformed to an external surface of the user 416. The transmitter system may conform to the user 416 without matching the external surface of the user 416 completely. That is, the housing shape may be adjusted to better match the external surface of the user 416 and to attempt to match different shapes of external surfaces of the user 416 or of different users. Further, transmitter systems discussed herein are preferably configured to have an adjustable length such that the transmitter systems may be adapted to or be used for different users of different sizes and/or shapes, or may be used for different portions of (e.g., with different sizes and/or shapes) a single user 416, with the transmitter systems being wrapped around and/or conformed to portions of one or more users that have different shapes, e.g., different perimeter contours and/or different perimeter lengths. Either or both of the transmitter systems 412, 414 may include a power supply, or may be connected to a power supply that is separate from the respective transmitter system 412, 414. A system that is disposed around a perimeter or axis need not be disposed around all 360° of the perimeter or axis. Similarly, a turn of a coil need not be disposed around all 360° of the perimeter or axis, but preferably extends substantially entirely around the perimeter or axis, e.g., being disposed over more than 330° of the perimeter or axis. Further, describing the system or coil as being disposed “around” an item such as a perimeter or axis does not require a particular shape, e.g., round/circular, of the item.

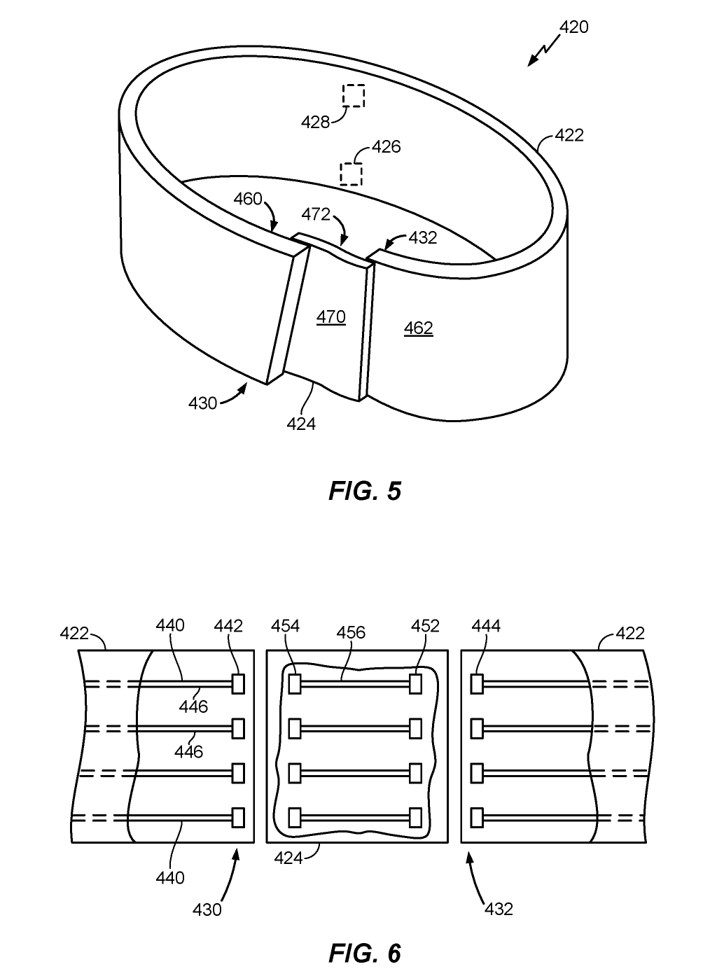

Referring to FIGS. 5-7, with further reference to FIGS. 1-4, a wireless charging device 420 includes a base 422 and an extension 424. The base 422 may be referred to as a housing, a substrate, or an enclosure. The base 422 is configured to be worn by the user 416. For example, the base 422 may be made of a flexible material such as a fabric, rubber, and/or other appropriate material(s). The device 420 is configured to produce a magnetic field, e.g., along an axis of portion of the user 416 around which the device 420 is disposed, e.g., wrapped. For example, the base 422 may be configured as a sheath to contain a coil that is configured to produce a magnetic field to transfer power wirelessly to a receiver, e.g., with the receiver being part of an implant disposed inside the user 416.

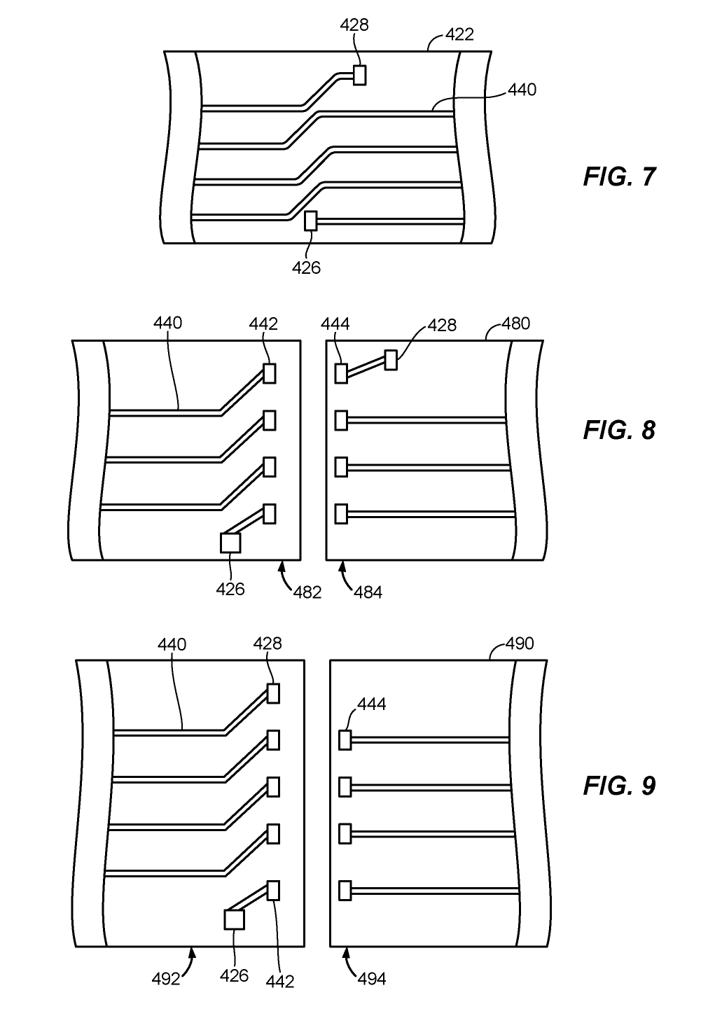

The base 422 contains a multi-turn coil 440 (not shown in FIG. 5) although the coil 440 may be accessible from outside of the base 422. The coil 440 is shaped and made of an electrically-conductive material to respond to receiving power to produce a magnetic field when a current is passed through the coil 440. An input terminal 426 of the coil 440 and a return terminal 428 of the coil 440 may be accessible from outside of the base 422. The device 420 is configured such that at least one turn of the coil 440 has an adjustable length, and preferably all or all but one or two of the turns (e.g., first and/or last turn) has an adjustable length. The terminals 426, 428 are connected to respective ends of the multi-turn coil 440 contained by the base 422, as best shown in FIG. 7. The terminals 426, 428 are configured to be coupled to a power supply. Each of the terminals 426, 428, also referred to as ports, may be, for example, a male or female connector that is accessible outside the base 422 for connection to a power supply that is external to the base 422. Alternatively, each of the terminals 426, 428 may be a pad that is electrically coupled to a power supply disposed internal to the base 422, or may be a pad that is electrically coupled to a connector that is accessible outside the base 422 for connection to a power supply that is external to the base 422. Alternatively, the terminals 426, 428 may be omitted and the coil 440 connected to a power supply that is disposed internally to the base 422. Still other configurations of the device 420 are possible.

The extension 424 may be selectively connected to intermediate ends 430, 432 of the base 422, and in particular to the coil 440 contained within the base 422, to adjust a length of the device 420. Further, multiple extensions may be connected in daisy-chain fashion and connected to the intermediate ends 430, 432 of the base 422 to provide different lengths of the device 420. The intermediate ends 430, 432 may be configured to connect to each other in various manners, and the extension 424 is configured to connect to the respective ends 430, 432 in the appropriate manners. Also, different extensions could have different lengths to help provide a selectable length of the device 420.

To enable connecting the base 422 to itself, the intermediate end 430 may include one or more connectors that can releasably mechanically connect and releasably electrically couple to one or more corresponding connectors of the intermediate end 432. For example, as shown, the intermediate end 430 includes male connectors 442 as shown in FIG. 6 electrically coupled to respective turns 446 of the multi-turn coil 440 contained in the base 422 and the intermediate end 432 includes female connectors 444 electrically coupled to respective turns 446 of the multi-turn coil 440 contained in the base 422. Similarly, to enable connecting the base 422 to the extension 424, the extension 424 includes female connectors 454 for connecting to the male connectors 442 of the intermediate ends 430 and includes male connectors 452 for connecting to the female connectors 444 of the intermediate end 432. The male connectors 442 of the extension 424 may be connected to female connectors 444 of another extension for use in a daisy chain of extensions.

As shown in FIG. 5, in this example, the extension 424 connects to a back side of the intermediate end 430 and to a front side of the intermediate end 432. Thus, the male connectors 442 of the base 422 are accessible through a back side 460 of the intermediate end 430 of the base 422 and the female connectors 444 are accessible through a front side 462 of the intermediate end 432 of the base 422. Similarly, the female connectors 454 are accessible through a front side 470 of the extension 424 and the male connectors 452 are accessible through a back side 472 of the extension 424. Further, as the extension 424 is configured to couple the intermediate ends 430, 432, the extension 424 has male connectors 452 and female connectors 454 electrically connected by electrical segments 456. The electrical segments 456 are coil-turn extension sections and are each a portion of a turn of the coil 440 when the extension 424 is coupled to the base 422 and thus each of the electrical segments 456 may be called an extension turn portion. The extension turn portion (or portions if multiple extensions are used) combines with a corresponding base turn portion of a turn of the coil 440 that is in the base 422 to complete a turn of the coil 440. Each of the electrical segments 456 is electrically coupled to (i.e., coupled to provide electricity to and/or receive electricity from) one of the connectors 452 and one of the connectors 454.

The extension 424 is preferably configured to adapt to a surface shape of the user 416. For example, the extension 424 may comprise a flexible sheath that contains the connectors 452, 454 and the electrical segments 456. Alternatively, the extension 424 may comprise a rigid housing that is shaped to accommodate a surface shape of the user 416, e.g., being curved to accommodate a curvature of an abdomen of the user 416, or being curved to accommodate a curvature of an arm of the user 416.

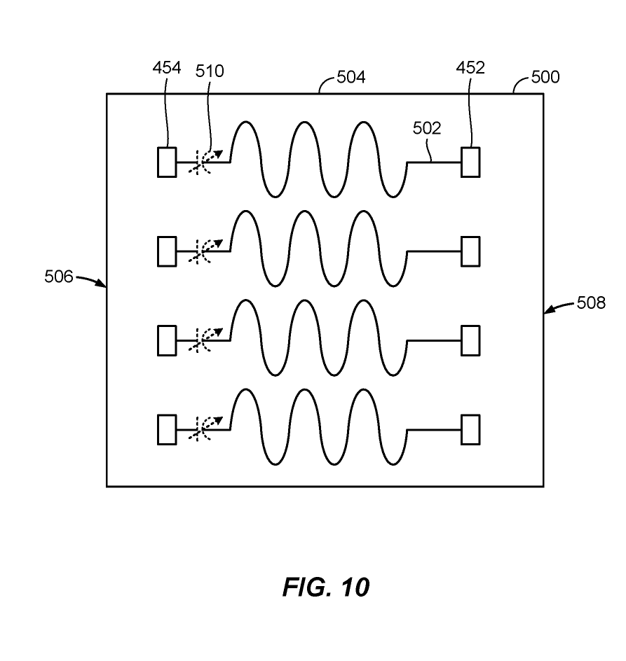

Alternative configurations of extensions are possible. For example, an extension may not only provide a length of conductor for one or more turns of the coil 440, the extension may provide a variable length of conductor for each of the one or more turns of the coil 440. For example, referring also to FIG. 10, an extension 500 includes electrical sections 502 that electrically couple the connectors 452 to the connectors 454, and that provide a variable turn portion length. The electrical sections 502 are flexible conductors, such as litz wires, that have a back-and-forth shape in a default state as shown in FIG. 10 and that are configured to be straightened (are able to be straightened). Further, such back-and-forth shapes may be provided in portions of the coil 440 contained in the base 422. A housing 504 of the extension 500 is stretchable such that ends 506, 508 of the extension 500 may be pulled away from each other. As the ends 506, 508 are pulled away from each other, the electrical sections 502 will straighten to provide a longer turn length. While a physical path length of the electrical sections 502 remains the same while the ends 506, 508 are pulled away from each other, the turn-length portions (e.g., horizontal lengths as shown) of the electrical sections 502 (and the physical separation of the connectors 452, 454) increase, thus increasing the turn lengths of the coil 440.

Other configurations of the base 422 and/or the extension 424 are possible. For example, referring to also FIGS. 8-9, bases may be configured to connect to each other near terminal ports of the bases. That is, instead of the bases being configured to connect to a power supply at terminal ports of a coil, and to be coupled to an extension at intermediate ends of the bases, the bases are configured to have terminal ends connect to each other or to one or more extensions. Referring to FIG. 8, a base 480 includes four of the male connectors 442 in an input end 482 and four female connectors 444 in a return end 484. Further, the input end 482 includes the input terminal 426 and the return end 484 includes the return terminal 428. As shown, in this configuration, the base 480 includes three turns of the coil 440. The configuration shown in FIG. 8 uses four male connectors and four female connectors, thus accommodating the extension 424 shown in FIG. 6. Referring to FIG. 9, a base 490 also accommodates the extension 424 but provides four turns of the coil 440. The base 490 includes a similar configuration of the input terminal 426 and a first of the male connectors 442, but has the return terminal 428 on an input end 492 as opposed to being disposed on an intermediate end 494. Still other configurations are possible. For example, a base may be used that is similar to the base 490 but with the input terminal 426 used instead of a bottommost one of the female connectors 444. In this case, an extension could have three sets of connectors and corresponding electrical segments, and a fourth turn of the coil 440 would be slightly shorter than the other turns if an extension is used. As another example, a base may be used that is similar to the base 480 but with the return terminal 428 used instead of a topmost one of the male connectors 442. In this case, an extension could use three sets of connectors and corresponding electrical segments, and a third turn of the coil 440 would be slightly shorter than the other turns if an extension is used.

Each of the turns in the multi-turn coil 440 has a length. For example, the length of a first turn may be from the input terminal 426 to where the coil is disposed above the input terminal 426, having passed through one of the male connectors 442 in the intermediate end 430 and one of the female connectors 444 in the intermediate end 432. The male connectors 442 in the input end 430, 482, 492 may be referred to as first turn ports and the female connectors 444 in the intermediate end 432, 484, 494 may be referred to as second turn ports. The female connectors 454 in the extension 424 may be referred to as first extension ports and the male connectors 452 in the extension 424 may be referred to as second extension ports. Thus, the first turn ports are configured to be selectively coupled to, or selectively decoupled from, the first extension ports and the second turn ports are configured to be selectively coupled to, or selectively decoupled from, the second extension ports. Similarly, the first turn ports are configured to be selectively coupled to, or selectively decoupled from, the second turn ports.

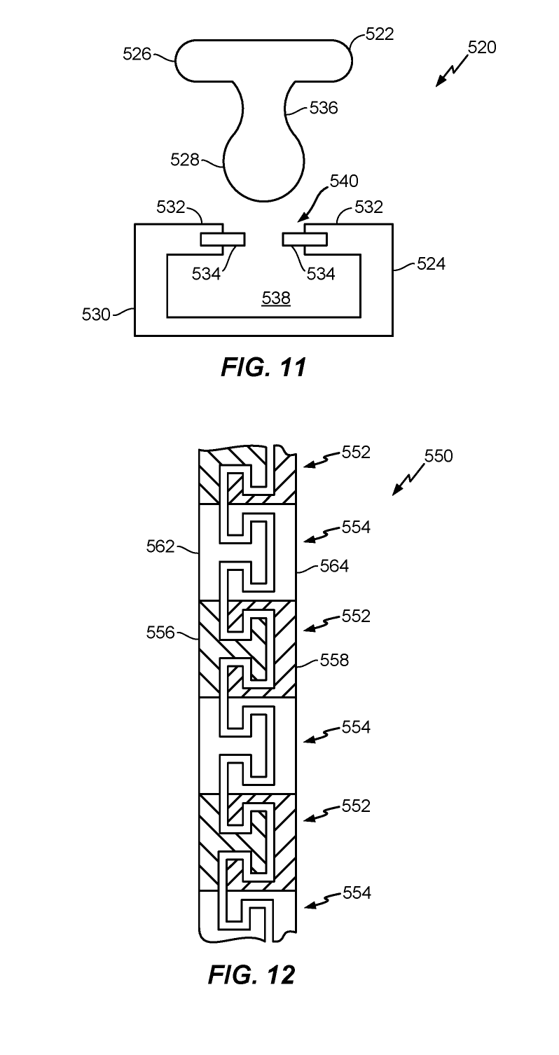

Various configurations of devices may be used to connect intermediate ends 430, 432 of the device 420 or to connect ends of the extension 424 to the intermediate ends 430, 432 of the device 420. For example, referring to FIG. 11, a snap 520 includes a male connector 522 and a female connector 524, which are examples of the male connector 442, 452 and the female connector 444, 454, respectively. The male connector 522 includes a head 526 and a bulb 528. The head 526 is configured to be pushed, e.g., by a thumb of a user to insert the male connector 522 into the female connector 524, in particular to insert the bulb 528 into the female connector 524. The female connector 524 includes a base 530, that includes a neck 532, and a valve 534. The valve 534 is configured to expand to accommodate the insertion of the bulb 528, and to constrict about a neck portion 536 of the male connector 522 to help retain the bulb 528 within a chamber 538 defined by the base 530 of the female connector 524. For example, the valve 534 may be spring-loaded such that the valve 534 may retract when pushed outwardly by the bulb 528 being inserted into the female connector 524 and constrict, i.e., close to reduce an opening 540 provided by the neck 532, after the bulb 528 enters the chamber 538 or after the bulb 528 is removed from the chamber 538. Thus, the valve 534 may partially close when the snap 520 is fully closed. Both the male connector 522 and the female connector 524 are at least partially electrically conductive to provide a low-ohmic connection of the male connector 522 to the female connector 524 for charging current to pass through the snap 520. For example, preferably at least an outer surface of the bulb 528 and an outer surface of the neck portion 536 of the male connector 522, and an external surface of the valve 534, are electrically conductive, e.g., being made of metal. Further, the valve 534 is preferably electrically coupled to the base 530, and the base 530 is electrically coupled to the coil 440, including to an electrical segment 456 of an extension. Similarly, the head 526 is preferably electrically coupled to the neck portion 536, and the head 526 is electrically coupled to the coil 440, including to an electrical segment 456 of an extension.

Referring to FIG. 12, another example of a connector configuration, here a zipper-style electrical connector 550, includes electrically conductive sections 552 and electrically insulating sections 554. Each of the electrically conductive sections 552 includes a male tooth 556 and a female receptacle 558. The male tooth 556 and the female receptacle 558 are both electrically conductive and coupled to the coil 440, including possibly to an electrical segment 456 of an extension. The electrically insulating sections 554 each include a male tooth 562 and a female receptacle 564. The male tooth 562 is shaped similarly to the male tooth 556 and the female receptacle 564 is shaped similarly to the female receptacle of 558. The male tooth 562 and the female receptacle 564 of the electrically insulating sections 554 are electrically insulating and nonconductive, and not coupled to the coil 440. While only one electrically insulating section 554 is shown separating electrically conductive sections 552, more than one electrically insulating section 554 may separate “adjacent” electrically conductive sections 552, i.e., electrically conductive sections 552 nearest each other but separated by at least one electrically insulating section 554. Further, while only one electrically conductive section 552 is shown for each electrically conductive portion of the zipper-style electrical connector 550, more than one electrically conductive section 552 may be used directly adjacent, and electrically coupled, to each other. The quantities and locations of the respective sections 552, 554 are disposed such that at least one of the electrically conductive male teeth 556 will make electrical contact with at least one of the electrically conductive female receptacles 558 in order to electrically couple different sections of the coil 440, or different sections of the coil 440 to different electrical segments 456 of an extension.

Further, combinations of connectors may be used. For example, one or more turns of the coil 440 may be connected to one or more other portions of the coil 440 or to one or more extension electrical segments using one or more of the snaps 520 while one or more other turns of the coil 440 may be connected to one or more other portions of the coil 440 or to one or more extension electrical segments using one or more portions of the connector 550. One or more other types of connectors may be used in combination with one or more of the connectors discussed herein. Preferably, however, a single type of connector is used in any one adjustable-length wireless power transmitter system for adjusting a coil length of the transmitter system.

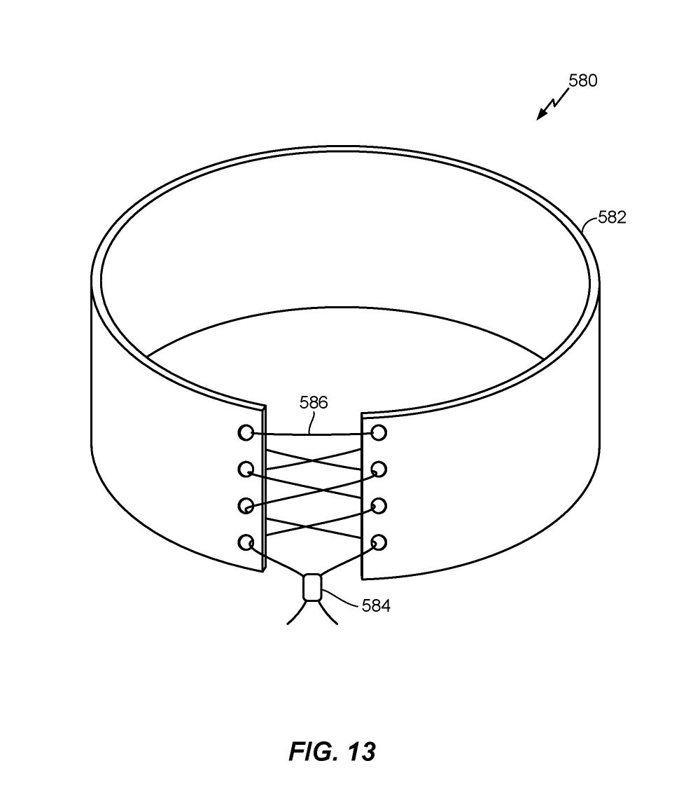

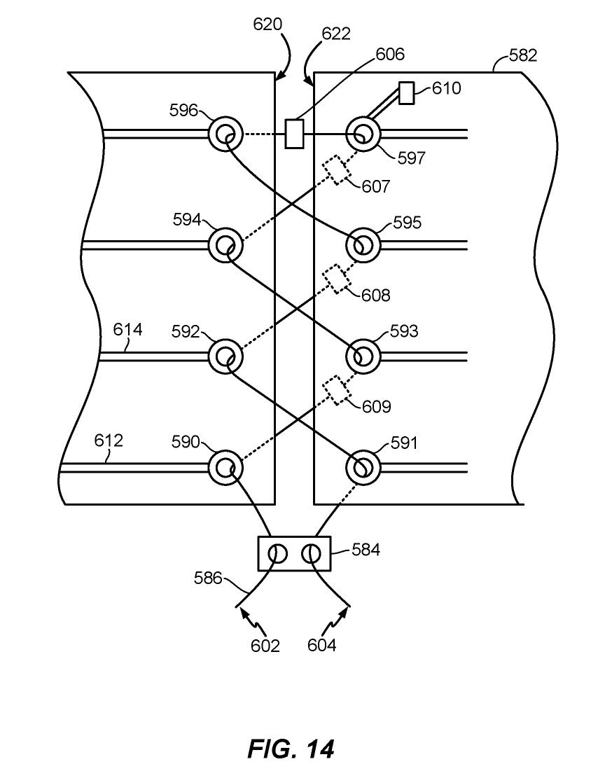

Other forms of adjustable-length wireless transmitter systems may be used. For example, referring to FIGS. 13-14, a wireless power transmitter system 580 includes an adjustable-length belt 582 and a cleat 584. The belt 582 is a corset-type adjustable length belt that includes multiple, here four, coil turns of a coil 581 for transmitting power wirelessly by magnetic coupling. A drawstring 586 may be loosened or tightened, i.e. the length of the drawstring 586 being threaded through the belt 582 being lengthened or shortened, to adjust, e.g., lengthen or shorten, the length of the transmitter system 580. The drawstring 586 is preferably made of an electrically-conducting material to provide portions of different turns of the coil 581. The cleat 584 is preferably made of an electrically non-conductive (i.e., at least poorly conducting) material and may be used to secure the drawstring 586 to hold ends 620, 622 of the belt 582 at a desired separation and thus retain the system 580 at a desired electrical length. The belt 582 includes conductive eyelets 590-597 through which the drawstring 586 is threaded and slidably connected (and electrically coupled). While eight eyelets 590-597 are shown, other quantities of eyelets may be used, e.g., particularly for coils with other than four turns. As shown, the drawstring 586 may be threaded through the eyelets 590-597 such that the drawstring 586 will be on one side of the belt 582 when crossing from left to right and be on the other side of the belt 582 when crossing from right to left to provide a separation between the segments of the drawstring 586, when the drawstring 586 crosses itself as shown, to help prevent shorting between the different sections of the drawstring 586. Further, and insulator such as an insulating bar (not shown) may be disposed between the ends 620, 622 of the belt 582 and between the different sections of the drawstring 586 to further isolate the sections of the drawstring 586 and further inhibit shorting between the sections of the drawstring 586.

Insulators are provided in the drawstring to help prevent shorts and help current be directed appropriately through the coil 581. The drawstring 586 includes an input end 602 coupled to a power supply (not shown) and a neutral end 604. An insulator 606 is disposed in the drawstring 586 between the eyelets 596, 597 that are disposed at an opposite end of the belt 582 from the eyelet 590 disposed nearest to the input end 602 of the drawstring 586 along the length of the drawstring 586. The insulator 606 helps prevent shorting of the eyelet 596 and the eyelet 597. Also or alternatively, the eyelet 597 may be electrically non-conducting, e.g., made of and/or coated with an electrically insulating material. The eyelet 597 is electrically coupled to an output port 610 that may be coupled to a return line of the power supply. Further, insulators 607-609 are disposed in the drawstring 586 to separate and help electrically isolate, and prevent electrically shorting of, respective pairs of the eyelets, in particular the eyelets 594, 597, the eyelets 592, 595, and the eyelets 590, 593, respectively. The insulator 607 may be eliminated, e.g., if the eyelet 597 is non-conducting, or at least does not electrically couple the drawstring 586 to the output port 610. The multi-turn coil 581 comprises N turns, with N>1 (here N=4), and the drawstring 586 comprises insulators disposed such that a first port, here the eyelet 596, of an N^{th} turn of the N turns is isolated from a second port, here the eyelet 597, of the N^{th} turn of the N turns and the second port (e.g., the eyelet 597, or 595, or 593) of an M^{th} turn of the N turns is isolated from the first port (e.g., the eyelet 594, 592, 590) of an (M−1)^{th} turn of the N turns where 0<M≤N (or where 0<M≤N−1). Thus, the belt 582, and in particular the eyelets 590-597, the output port 610, with the drawstring 586, including the isolators 606-609, and the cleat 584 are configured to receive current from the input end 602 of the drawstring 586 and conduct this current to the output port 610. The current will flow from the input end 602 of the drawstring 586 to the eyelet 590, through a coil turn 612 to the eyelet 591, through the drawstring 586 to the eyelet 592, through a coil turn 614 to the eyelet 593, etc., until the current reaches the output port 610.

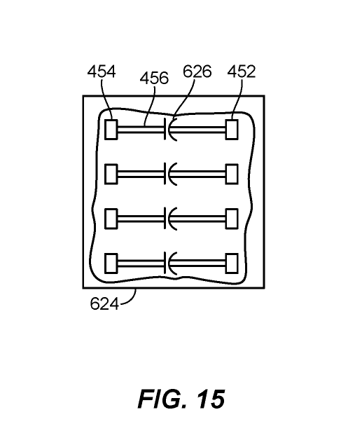

For each different length (i.e., electrical length) of the transmitter coil, e.g., the transmitter coil 440, the length may be determined and appropriate impedance tuning performed. For example, the controller 240 (FIG. 2) may be configured to determine an electrical length of the transmitter coil 440 and to tune a resonant circuit (e.g., adjust a capacitance, etc.) to accommodate for the length of the transmitter coil. Different electrical lengths of the transmitter coil 440 will have different inductances and thus tuning may be performed based on the different inductances, e.g., to maintain a desired resonant frequency. Various configurations are possible for tuning the resonant circuit. For example, one or more variable capacitors may be provided along the length of the transmitter coil 440, and/or in parallel with the transmitter coil 440, and one or more capacitance values of the one or more variable capacitors changed in accordance with the length of the transmitter coil 440. For example, as shown in FIG. 10, one or more variable capacitors 510 may be provided in the electrical segments 502 (and/or in portions of the transmitter coil 440 in the base 422). The variable capacitors 510 may be communicatively coupled to the controller 240. The capacitance values of the variable capacitors 510 may be changed, e.g., by the controller 240, based on the length of the transmitter coil 440 (e.g., based on the separation of the connectors 452, 454 or the change in length of the base 422). As another example, referring also to FIG. 15, an extension 624 may be provided that is configured similarly to the extension 424 but that includes a capacitor 626 disposed in each of the electrical segments 456. The capacitors 626 may have different values or substantially the same value (e.g., within 5% of each other). Further, different extensions 624 may have different sizes, with different lengths of the electrical segments 456), and different capacitance values of the capacitors 626. Using one or more of the extensions 624 may reduce voltage and circulating current, which may be useful in large-antenna applications.

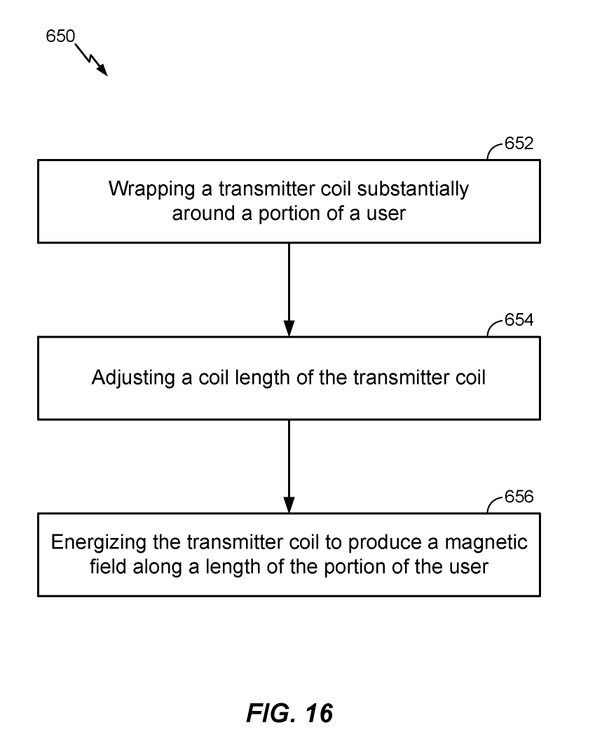

Referring to FIG. 16, with further reference to FIGS. 1-14, a method 650 of providing wireless power to an implant includes the stages shown. The method 650 is, however, an example only and not limiting. The method 650 may be altered, e.g., by having stages added, removed, rearranged, combined, performed concurrently, and/or having single stages split into multiple stages.

At stage 652, the method 650 includes wrapping a transmitter coil substantially around a portion of a user. For example, the belt transmitter system 412 and/or the arm cuff transmitter system 414 can be wrapped around a torso, an arm, or another body portion, of the user 416. The transmitter system 412, 414 may be disposed about a portion of the user 416, possibly being disposed entirely around a perimeter of the body portion, or possibly being disposed around less than an entire perimeter of the body portion. Also or alternatively, even if the transmitter system 412, 414 is disposed around an entire perimeter of the body portion of the user 416, at least some of a wireless power transmitting portion of the transmitter system 412, 414 may be disposed about less than the entire perimeter of the body portion.

At stage 654, the method 650 includes adjusting a coil length of the transmitter coil. A transmitter coil of the transmitter system 412, 414 is adjusted, e.g., to accommodate different sizes of users and/or different portions of the user 416, to be approximately the length of the perimeter of the body portion about which the transmitter system 412, 414 is disposed. For example, one or more extensions 424 may be selectively used in the device 420 to extend or contract a length of the transmitter coil 440 of the device 420 to best approximate a perimeter length of the body portion about which the device 420 is placed. Thus, adjusting of a coil length of a transmitter may comprise, for example, separating respective portions of each of multiple turns of a transmitter coil from each other, inserting an extension between the respective portions of each of the multiple turns of the transmitter coil, and electrically coupling the respective portions of each of multiple turns of the transmitter coil to each other through the extension. Further, an extension itself may be lengthened, e.g., an effective coil length of the extension 500 may be lengthened by pulling ends 506, 508 away from each other to lengthen electrical sections 502 that have a back-and-forth shape absent a pulling or straightening force. That is, absent a force to overcome a bias of the electrical segments 502 to a resting state, e.g., having a zig-zag shape. With the appropriate extension(s) in place, the extension(s) is(are) coupled to the transmitter coil (including to another extension as appropriate), for example using the snap 520 and/or the connector 550. As another example, the drawstring 586 of the wireless power transmitter system 580 may be tightened (e.g., pulled) or loosened to adjust the length of a coil of the system 580 to accommodate a body portion about which the belt 582 is wrapped. Thus, the drawstring 586 may be loosened or cinched to adapt to a perimeter of the user 416, e.g., to fit snugly about a body portion of the user 416. Once adjusted, the drawstring 586 is preferably inhibited from further movement using the cleat 584 to help prevent unintended movement of the drawstring 586.

At stage 656, the method 650 includes energizing the transmitter coil to produce a magnetic field along a length of the portion of the user. With the transmitter coil appropriately adjusted, current may be supplied to the coil from a power supply to induce a magnetic field for magnetically coupling power to a receiver. For example, with the device 420 disposed about a waist of the user 416, current may be supplied to the input terminal 426 such that current flows through the coil 440 to the return terminal 428, producing a magnetic field directed along the torso of the user 416. As another example, current may be supplied through the input end 602 of the drawstring 586 such that current flows through the drawstring 586, the eyelets 590-596, coil segments, to the output port 610, and from the output port 610 to the power supply to produce a magnetic field.

Other stages and/or features may be added to the method 650. For example, the method 650 may include determining a length of a transmitter coil, and tuning an impedance accordingly. For example, the controller 240 may determine an electrical length of the transmitter coil 440 and tune a resonant circuit to accommodate for the length of the transmitter coil. As another example, one or more capacitances may be added to the transmitter coil, e.g., by adding one or more of the extensions 624 that include the capacitors 626. These are examples of the method 650 including adjusting a capacitance coupled to the transmitter coil 440 responsive to adjusting a turn length of the transmitter coil 440.

Other Considerations

Other examples and implementations are within the scope and spirit of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, “or” as used in a list of items prefaced by “at least one of” or prefaced by “one or more of” indicates a disjunctive list such that, for example, a list of “at least one of A, B, or C,” or a list of “one or more of A, B, or C” means A or B or C or AB or AC or BC or ABC (i.e., A and B and C), or combinations with more than one feature (e.g., AA, AAB, ABBC, etc.).

Further, an indication that information is sent or transmitted, or a statement of sending or transmitting information, “to” an entity does not require completion of the communication. Such indications or statements include situations where the information is conveyed from a sending entity but does not reach an intended recipient of the information. The intended recipient, even if not actually receiving the information, may still be referred to as a receiving entity, e.g., a receiving execution environment. Further, an entity that is configured to send or transmit information “to” an intended recipient is not required to be configured to complete the delivery of the information to the intended recipient. For example, the entity may provide the information, with an indication of the intended recipient, to another entity that is capable of forwarding the information along with an indication of the intended recipient.

Substantial variations may be made in accordance with specific requirements. For example, customized hardware might also be used. Further, connection to other computing devices such as network input/output devices may be employed.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, structures, and techniques have been shown without unnecessary detail in order to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional stages or functions not included in the figure.

Components, functional or otherwise, shown in the figures and/or discussed herein as being coupled, connected, or communicating with each other are operably coupled. That is, they may be directly or indirectly, wired or wirelessly, connected to enable signal flow between them.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of operations may be undertaken before, during, or after the above elements are considered. Accordingly, the above description does not bound the scope of the claims.

Further, more than one invention may be disclosed.

1. A wireless charging device comprising:
a base configured to be worn by a user; and
a coil attached to the base and comprising an electrically conductive material shaped to produce a magnetic field to transmit power wirelessly to a receiver in response to receiving power, the coil including multiple turns each having a turn length with at least one of the multiple turns having an adjustable turn length, the multiple turns being disposed along an axis such that each of the multiple turns is disposed around the axis for the respective turn length of the turn.

2. The wireless charging device of claim 1, wherein each of the multiple turns includes a base turn portion and is electrically coupled to a first turn port and a second turn port, and wherein the wireless charging device further comprises an extension comprising multiple electrical segments each being an extension turn portion and electrically coupled to a first extension port and a second extension port, wherein for each combination of one of the base turn portions and one of the extension turn portions, the first turn port and the first extension port are configured to be selectively coupled to each other or selectively decoupled from each other, and the second turn port and the second extension port are configured to be selectively coupled to each other or selectively decoupled from each other.

3. The wireless charging device of claim 2, wherein the base comprises a first flexible sheath that contains the coil and that is configured to conform to a person, and wherein the extension comprises a second flexible sheath that contains the multiple electrical segments.

4. The wireless charging device of claim 3, wherein each of the multiple electrical segments includes a back-and-forth shape along at least a portion of the electrical segment that is configured to be straightened such that a distance between the first extension port and the second extension port increases.

5. The wireless charging device of claim 2, wherein for each combination of one of the base turn portions and one of the extension turn portions, one the first turn port or the first extension port comprises a male connector, and the other one of the first turn port or the first extension port comprises a female connector.

6. The wireless charging device of claim 5, wherein the base and the extension are configured to provide a zipper-style connector to connect the base turn portions and the extension turn portions, the zipper-style connector having alternating electrically inductive and electrically insulating sections.

7. The wireless charging device of claim 2, wherein at least one of the multiple electrical segments includes a capacitor.

8. The wireless charging device of claim 1, wherein each of the multiple turns is electrically coupled to a first turn port and a second turn port, the coil further comprising a conductive drawstring threaded through each of the first turn ports and the second turn ports.

9. The wireless charging device of claim 8, wherein the multiple turns comprise N turns, with N>1, and wherein the coil comprises a plurality of insulators disposed such that the first turn port of an N^{th} turn of the multiple turns is isolated from the second turn port of the N^{th} turn of the multiple turns and the second turn port of an M^{th} turn of the multiple turns is isolated from the first turn port of an (M−1)^{th} turn of the multiple turns where 0<M≤N.

10. The wireless charging device of claim 1, wherein each of the multiple turns includes a back-and-forth shape along at least a portion of the turn that is configured to be straightened such that the adjustable turn length increases.

11. The wireless charging device of claim 10, further comprising:
a variable capacitor in at least one of the multiple turns; and
a controller communicatively coupled to the variable capacitor and configured to adjust a capacitance value of the variable capacitor based on the adjustable turn length.

12. A wireless charging device comprising:
transmitting means for wirelessly transmitting power, the transmitting means including an input port and a return port, the input port and the return port being configured to electrically couple to a power source; and
housing means for housing the transmitting means and for positioning the transmitting means around of a first portion of a first user's body of a first perimeter length;
wherein the transmitting means are further for extending a turn length of a conductor coupling the input port to the return port for the transmitting means to be positioned around a second portion of a second user's body of a second perimeter length that is greater than the first perimeter length.

13. The wireless charging device of claim 12, wherein the transmitting means have a first intermediate end and a second intermediate end, and wherein the transmitting means include extending means for electrically coupling the first intermediate end to the second intermediate end while extending the turn length of the conductor coupling the input port to the return port.

14. The wireless charging device of claim 13, wherein the first intermediate end is for selectively coupling to the second intermediate end.

15. The wireless charging device of claim 14, wherein the transmitting means comprise a multi-turn coil and wherein the extending means comprise a plurality of coil-turn extension sections each comprising a first extension end configured to releasably couple to the first intermediate end and a second extension end configured to releasably couple to the second intermediate end.

16. The wireless charging device of claim 15, wherein the housing means comprise a first flexible sheath that contains a base portion of the multi-turn coil.

17. The wireless charging device of claim 16, wherein the extending means comprise a second flexible sheath that contains the plurality of coil-turn extension sections.

18. The wireless charging device of claim 15, wherein each of the plurality of coil-turn extensions includes a back-and-forth shape along at least a portion of the coil-turn extension that is configured to be straightened.

19. The wireless charging device of claim 13, wherein the transmitting means comprise a multi-turn coil, wherein the first intermediate end comprises a first intermediate port for each turn of the multi-turn coil and the second intermediate end comprises a second intermediate port for each turn of the multi-turn coil, and wherein the transmitting means further comprise a conductive drawstring slidably connected to each of the first intermediate ports and the second intermediate ports.

20. The wireless charging device of claim 19, wherein the multi-turn coil comprises N turns, with N>1, and wherein the transmitting means comprise a plurality of insulators disposed such that the first intermediate port of an N^{th} turn of the N turns is isolated from the second intermediate port of the N^{th} turn of the N turns and the second intermediate port of an M^{th} turn of the N turns is isolated from the first intermediate port of an (M−1)^{th} turn of the N turns where 0<M≤N.

21. The wireless charging device of claim 13, wherein the extending means comprise at least one capacitor.

22. The wireless charging device of claim 13, wherein the extending means comprise a zipper-style connector having alternating conductive and insulating sections.

23. The wireless charging device of claim 12, wherein the transmitting means comprise capacitance means for changing a capacitance of the transmitting means in accordance with the turn length of the conductor.

24. The wireless charging device of claim 23, wherein the capacitance means comprises at least one variable capacitor.

25. A method of providing wireless power to an implant, the method comprising:
wrapping a transmitter coil substantially around a portion of a user;
adjusting a turn length of the transmitter coil; and
energizing the transmitter coil to produce a magnetic field along a length of the portion of the user.

26. The method of claim 25, wherein adjusting the turn length of the transmitter coil comprises separating respective portions of each of multiple turns of the transmitter coil from each other, inserting an extension between the respective portions of each of the multiple turns of the transmitter coil, and electrically coupling the respective portions of each of the multiple turns of the transmitter coil to each other through the extension.

27. The method of claim 25, wherein adjusting the turn length of the transmitter coil comprises tightening a conductive drawstring to shorten the turn length of the transmitter coil.

28. The method of claim 25, wherein adjusting the turn length of the transmitter coil comprises loosening a conductive drawstring to lengthen the turn length of the transmitter coil.

29. The method of claim 25, further comprising adjusting a capacitance coupled to the transmitter coil responsive to adjusting the turn length of the transmitter coil.
